(19) United States
(12) Patent Application Publication
(10) Pub. No.: US 2020/0113797 A1
(43) Pub. Date: Apr. 16, 2020

(54) EMULSIONS HAVING (POLY)HYDROXYCARBOXYLIC ACIDS, MONO- OR DISTEARATE, AND STARCH

(71) Applicant: BEIERSDORF AG, Hamburg (DE)

(72) Inventors: Susanne MORGENROTH-EHRICH, Norderstedt (DE); Silke KOHLHASE, Pinneberg (DE); Anette BUERGER, Hamburg (DE)

(21) Appl. No.: 16/623,466

(22) PCT Filed: May 22, 2018
(86) PCT No.: PCT/EP2018/063334

§ 371 Date: Dec. 17, 2019

(30) Foreign Application Priority Data
Jun. 21, 2017 (DE) 10 2017 210 389.2

(51) Int. Cl. A61K 8/37; A61K 8/06; A61K 8/365; A61K 8/36; A61K 8/73; A61K 8/362; A61K 8/49; A61K 8/86; A61Q 19/00; A61Q 19/08; A61Q 19/02

(52) U.S. Cl. CPC A61K 8/37; A61K 8/062; A61K 8/365; A61K 8/361; A61K 8/732; A61Q 19/02; A61K 8/498; A61K 8/86; A61Q 19/007; A61Q 19/08; A61K 8/362

(57) ABSTRACT

Cosmetic or dermatological O/W emulsions, having a content of a) 3-10 wt % of one or more α-hydroxycarboxylic acids and/or one or more β-hydroxycarboxylic acids and/or one or more polyhydroxy acids, b) one or more distearates having a melting range in the temperature range of 53-64° C., c) one or more monostearates having a melting range in the temperature range of 40-57° C., d) a water phase, e) an oil phase, f) a content of 0.1-2 wt % of one or more starches pre-swollen at 85-95° C. in an aqueous medium, the percentage specifications being relative to the total weight of the preparations.

The present invention relates to cosmetic preparations having a content of one or more α-hydroxycarboxylic acids and/or one or more β-hydroxycarboxylic acids and/or one or more polyhydroxy acids, one or more distearates, and starch.

Many personal care products currently available to consumers are aimed primarily at improving the health or external appearance of the skin or of the hair. Among skincare products, many are aimed at slowing, minimizing or even eliminating the formation of skin wrinkles and other histological changes that are generally associated with the aging of the skin or with skin damage due to environmental effects.

Numerous compounds have been described in the prior art as being suitable for controlling the state of the skin, including controlling fine lines, wrinkles and other forms of uneven or rough surface condition associated with aged or photodamaged skin.

The skin is subject to stresses from numerous external and internal factors. Extrinsic factors include ultraviolet radiation (e.g. sunlight), environmental pollution, wind, heat, low humidity, aggressive surfactants, abrasives and the like.

Intrinsic factors include chronological age and other biochemical changes within the skin. Irrespective of whether these factors are external or internal, they result in visible signs of the aging of the skin and damage from environmental effects, such as the development of wrinkles and other forms of roughness (including enlarged pores, desquamation, and skin lines) and also other histological changes associated with aging of or damage to the skin. Skin wrinkles are to many people a reminder of the loss of youth.

A further aim of skincare is to compensate for the loss of oils and water from the skin caused by daily washing. This becomes particularly important if the natural capacity for regeneration is inadequate. In addition, skincare products need to protect against environmental effects, in particular sun and wind, and slow the aging of the skin.

Chronological aging of the skin is caused for example by endogenous, genetically deter-mined factors. In the epidermis and dermis, aging results, for example, in the following structural damage and functional impairment, which can also be grouped together under the term “senile xerosis”:

a) dryness, roughness, and development of fine wrinkles caused by dryness,
b) itching, and
c) decreased refatting by sebaceous glands (for example after washing).

Exogenous factors, such as UV light and chemical toxins, can have a cumulative effect and, for example, accelerate the endogenous aging process or add to it. In the epidermis and dermis, exogenous factors result, for example, in the following structural damage and functional impairment in the skin, which exceed the damage from chronological aging in both extent and nature:

d) visible vascular dilations (telangiectasias, couperose);
e) sagging skin and development of wrinkles;
f) local hyper-, hypo-, and dyspigmentation (for example age spots), and
g) increased susceptibility to mechanical stress (for example cracking).

The present invention relates especially to products for caring for naturally aged skin and for treating the secondary damage from photoaging, in particular the phenomena mentioned under a) to g).

Skin unevenness, for example minor wrinkles, is a particularly unwelcome phenomenon that accompanies the aging of the skin. One of the aims of cosmetic compositions is to eliminate, cover up, and/or minimize such phenomena. One way of achieving this consists of supplementing the skin with care components or active substances, but this can be lengthy and tedious.

α-Hydroxycarboxylic acids are well known in cosmetics and in dermatological formulations. They are reputed to smoothen skin wrinkles and are also known as anti-acne agents.

In acne-prone skin, flakes of old skin are removed, thereby enhancing the appearance of the skin and reducing blemishes. The skin is able to renew itself more readily.

It can be problematic to formulate appropriate products that contain more than 3% by weight of α-hydroxycarboxylic acids, since these often have unappealing sensory properties or poor skin tolerability—or both. If a high concentration of free α-hydroxycarboxylic acids, 1-5% for example, needs to be ensured in the product, the pH must be in the range between about 3.6 and 4.0. However, this in turn means it is also difficult to formulate a stable emulsion.

The object of the present invention was therefore to address these problems.

This object was achieved by

Cosmetic or dermatological O/W emulsions having a content of
a) 3-10% by weight of one or more α-hydroxycarboxylic acids and/or one or more β-hydroxycarboxylic acids and/or one or more polyhydroxy acids,
b) one or more distearates having a melting range in the temperature range of 53-64° C.,
c) one or more monostearates having a melting range in the temperature range of 40-57° C.,
d) a water phase
e) an oil phase
f) a content of 0.1-2% by weight of one or more starches preswollen at 85-95° C. in an aqueous medium,
   wherein the percentages are based on the total weight of the preparations.

Such preparations are particularly suitable for treating and/or preventing skin damage caused by UV light and the effects of skin aging or for treating pigmentation disorders and/or for skin lightening. It is also possible to use them for treating acne and desquamation.

The invention also provides a process for producing O/W emulsions, characterized in that mixtures of
a) 3-10% by weight of one or more α-hydroxycarboxylic acids and/or one or more p-hydroxycarboxylic acids and/or one or more polyhydroxy acids,
b) one or more distearates having a melting range in the temperature range of 53-64° C.,
c) one or more monostearates having a melting range in the temperature range of 40-57° C.,
d) a content of 0.1-2% by weight of one or more starches preswollen at 85-95° C. in an aqueous medium,
e) are combined, wherein the water-soluble or hydrophilic constituents are dissolved or dispersed in
f) a water phase
g) and the oil-soluble or lipophilic constituents are dissolved or dispersed in
h) an oil phase,
   and the oil phase and the water phase are emulsified together optionally with heating and with stirring.

The α-hydroxycarboxylic acid or acids are advantageously selected from the compounds of the general formula

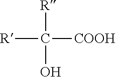

and the β-hydroxycarboxylic acid or acids are advantageously selected from the compounds of the general formula

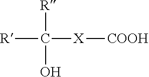

wherein X represents an aliphatic CH₂ group, a cycloaliphatic CH group, an aromatic CH group or a CH(OH) group,
wherein R′ and R″ are each independently selected from the group
(a1) H—,
(a2) branched or unbranched C₁₋₂₅ alkyl,
(a3) branched or unbranched C₁₋₂₅ alkyl substituted with one or more carboxyl groups and/or hydroxyl groups and/or aldehyde groups and/or oxo groups (keto groups)
(a4) phenyl,
(a5) phenyl substituted with one or more carboxyl groups and/or hydroxyl groups and/or branched and/or unbranched C₁₋₂₅ alkyl groups,
or wherein the α-carbon atom of the α-hydroxycarboxylic acid or the β-carbon atom of the β-hydroxycarboxylic acid form together with R′ and X an
(a6) unsubstituted cycloalkyl group having 3 to 7 ring atoms or a
(a7) cycloalkyl group having 3 to 7 ring atoms and substituted with one or more carboxyl groups and/or hydroxyl groups and/or oxo groups (keto groups) and/or branched and/or unbranched C₁₋₂₅ alkyl groups,
   and
   wherein the α-hydroxycarboxylic acid or the α-hydroxycarboxylic acids or the β-hydroxycarboxylic acid or the β-hydroxycarboxylic acids may optionally be present in the form of their physiologically tolerated salts and/or ethyl esters and/or methyl esters.

Preferred α-hydroxycarboxylic acids are glycolic acid, lactic acid, and citric acid, the preferred β-hydroxycarboxylic acid is salicylic acid, and the preferred polyhydroxy acid is gluconolactone.

The preferred distearate is PEG-150 distearate.

Preferred monostearates are glyceryl stearate and PEG-40 stearate.

The cosmetic or dermatological preparations according to the invention may have the customary composition, and be used in the treatment, care, and cleansing of the skin and/or hair and as a makeup product in decorative cosmetics. They preferably contain 5 to 10% by weight, based on the total weight of the preparations, of one or more α-hydroxycarboxylic acids and/or one or more β-hydroxycarboxylic acids.

The cosmetic or dermatological preparations according to the invention preferably contain 0.001% by weight to 10% by weight, more preferably 0.05% by weight to 5% by weight, particularly preferably 0.1-2.0% by weight, based on the total weight of the preparations, of one or more distearates that have a melting range in the temperature range of 53-64° C.

The cosmetic or dermatological preparations according to the invention preferably contain 0.001% by weight to 10% by weight, more preferably 0.05% by weight to 5% by weight, particularly preferably 0.1-2.0% by weight, based on the total weight of the preparations, of one or more monostearates, which have a melting range in the temperature range of 40-57° C.

Inventive emulsions in the context of the present invention, for example in the form of a cream, a lotion or a cosmetic milk, are advantageous and contain, for example, fats, oils, waxes and/or other fatty substances, and also water and one or more emulsifiers as are customarily used for formulations of this type.

It also possible and advantageous in the context of the present invention to add the active substance used according to the invention to aqueous systems or surfactant preparations for cleansing the skin and the hair.

It is of course known to those skilled in the art that sophisticated cosmetic compositions are generally not conceivable without the customary auxiliaries and additives. Examples of these include consistency regulators, fillers, fragrance, colorants, emulsifiers, additional active substances such as vitamins or proteins, light protectants, stabilizers, insect repellents, alcohol, water, salts, antimicrobially, proteolytically or keratolytically active substances, etc.

Mutatis mutandis, corresponding requirements apply to the formulation of medicinal products.

Topical medicinal compositions in the context of the present invention generally comprise one or more medicaments at an effective concentration. For the sake of simplicity and in order to make a clear distinction between cosmetic and medicinal use and corresponding products, reference is made to the statutory provisions of the Federal Republic of Germany (e.g. Kosmetikverordnung [Cosmetics Ordinance], Lebensmittel-und Arzneimittelgesetz [Food and Medicinal Products Act]).

Here it is also advantageous to include the active substance employed according to the invention as an additive in preparations that already contain other active substances for other purposes.

The cosmetic and/or dermatological formulations according to the invention may have the customary composition, and be used in the treatment of the skin and/or hair in the sense of a dermatological treatment or of a treatment in the sense of care cosmetics. They may however also be used in makeup products in decorative cosmetics or in cosmetic and dermatological cleansing products.

Where the cosmetic or dermatological preparation is a solution or lotion, it is possible to use as solvent: water or aqueous solutions, also oils such as triglycerides of capric or caprylic acid, but preferably castor oil, fats, waxes, and other natural and synthetic fatty substances, preferably esters of fatty acids with alcohols having a low number of carbon atoms, for example with isopropanol, propylene glycol or glycerol, or esters of fatty alcohols with alkanoic acids having a low number of carbon atoms or with fatty acids, but also alcohols, diols or polyols having a low number of carbon atoms and the ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether, and analogous products.

Mixtures of the abovementioned solvents are used in particular, for example water may be an advantageous further constituent in the case of alcoholic solvents.

The active substances according to the invention may also be used particularly advantageously in microemulsions, for example as described in German patent application DE-195 9 079.

The oil phase of the preparations according to the invention is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group consisting of esters of aromatic carboxylic acids and saturated and/or unsaturated, branched and/or unbranched alcohols having a chain length of 3 to 30 C atoms. Such ester oils may then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and also synthetic, semisynthetic, and natural mixtures of such esters, for example jojoba oil.

In addition, the oil phase may advantageously be selected from the group of the branched and unbranched hydrocarbons and hydrocarbon waxes, silicone oils, dialkyl ethers, the group consisting of saturated or unsaturated, branched or unbranched alcohols, and also fatty acid triglycerides, namely triglyceryl esters of saturated and/or unsaturated, branched, and/or unbranched alkanecarboxylic acids having a chain length of 8 to 24, especially 12-18 C atoms. The fatty acid triglycerides may for example advantageously be selected from the group of synthetic, semisynthetic, and natural oils, e.g. olive oil, sunflower oil, soybean oil, groundnut oil, rapeseed oil, almond oil, palm oil, coconut oil, palm kernel oil, and other similar oils.

Any mixtures of such oil and wax components may also be used advantageously in the context of the present invention.

The oil phase is advantageously selected from the group 2-ethylhexyl isostearate, octyldodecanol, isotridecyl isononanoate, isoeicosane, 2-ethylhexyl cocoate, C12-15 alkyl benzoate, capryl/capric triglyceride, dicaprylyl ether.

Particularly advantageous are mixtures of C12-15 alkyl benzoate and 2-ethylhexyl isostearate, mixtures of C12-15 alkyl benzoate and isotridecyl isononanoate, and mixtures of C12-15 alkyl benzoate, 2-ethylhexyl isostearate, and isotridecyl isononanoate.

Of the hydrocarbons, paraffin oil, squalane, and squalene may be used advantageously in the context of the present invention.

The oil phase may advantageously further include a content of cyclic or linear silicone oils or consist entirely of such oils, although it is preferable to employ an additional content of other oil phase components besides the silicone oil or silicone oils.

Cyclomethicone (octamethylcyclotetrasiloxane) is advantageously employed as the silicone oil to be used according to the invention. However, other silicone oils are also advantageous for the purposes of the present invention, for example hexamethylcyclotrisiloxane, polydime-thylsiloxane, poly(methylphenylsiloxane).

Particularly advantageous are, moreover, mixtures of cyclomethicone and isotridecyl isononanoate and of cyclomethicone and 2-ethylhexyl isostearate.

The content of the oil phase is advantageously between 1 and 50% by weight based on the total weight of the preparations, preferably 2.5 to 30% by weight, more preferably 5 to 15% by weight.

The cosmetic and/or dermatological formulations according to the invention are used by ap-plying a sufficient amount to the skin in the manner customary for cosmetics and dermatological agents.

The examples below are intended to illustrate the invention without limiting it.

| Example 1 | % by weight |
|---|---|
| Panthenol + Aqua | 0.800 |
| Glycyrrhiza inflata root extract | 0.050 |
| Dimethicone | 5.000 |
| Glyceryl stearate | 3.000 |
| PEG-40 stearate | 2.000 |
| Tapioca starch + Aqua | 4.000 |
| Glycerol + Aqua | 5.000 |
| Lactic acid + Aqua | 2.220 |
| Aqua + sodium hydroxide | 5.400 |
| Glycolic acid + Aqua | 10.714 |
| Salicylic acid | 0.500 |
| Phenoxyethanol | 0.300 |
| Cetearyl alcohol | 3.000 |
| Xanthan gum | 0.500 |
| PEG-150 distearate | 1.000 |
| Aqua + trisodium EDTA | 1.000 |
| Pigments/colorants | As required |
| Water | To 100.000 |

| Example 2 | % by weight |
|---|---|
| Panthenol + Aqua | 0.800 |
| Carnitine | 0.500 |
| Glycyrrhiza inflata root extract | 0.050 |
| Dimethicone | 5.000 |
| Glyceryl stearate | 3.000 |
| PEG-40 stearate | 2.000 |
| Tapioca starch + Aqua | 4.000 |
| Glycerol + Aqua | 5.000 |
| Lactic acid + Aqua | 2.220 |
| Aqua + sodium hydroxide | 5.400 |
| Glycolic acid + Aqua | 10.714 |
| Salicylic acid | 0.500 |
| Phenoxyethanol | 0.300 |
| Cetearyl alcohol | 3.000 |
| Xanthan gum | 0.500 |
| PEG-150 distearate | 1.000 |
| Aqua + trisodium EDTA | 1.000 |
| Pigments/colorants | As required |
| Water | 100.000 |

| Example 3 | % by weight |
|---|---|
| Panthenol + Aqua | 0.800 |
| Isobutylamido Thiazolyl Resorcinol | 0.200 |
| Glycyrrhiza inflata root extract | 0.050 |
| Dimethicone | 5.000 |
| Glyceryl stearate | 3.000 |
| PEG-40 stearate | 2.000 |
| Tapioca starch + Aqua | 4.000 |
| Parfum | 0.250 |
| Glycerol + Aqua | 5.000 |
| Aqua + sodium hydroxide | 5.100 |
| Gluconolactone | 2.000 |
| Glycolic acid + Aqua | 10.000 |
| Salicylic acid | 1.000 |
| Pentanediol | 0.500 |
| Phenoxyethanol | 0.300 |
| Cetearyl alcohol | 3.000 |
| Acrylates/C10-30 Alkyl Acrylate | 0.150 |
| Crosspolymer | |
| Xanthan gum | 0.500 |
| PEG-150 distearate | 1.000 |
| Aqua + trisodium EDTA | 1.000 |
| Pigments/colorants | As required |
| Water | 100.000 |

| Example 4 | % by weight |
|---|---|
| Panthenol + Aqua | 0.800 |
| Carnitine | 0.500 |
| Glycyrrhiza inflata root extract | 0.050 |
| Isodecyl neopentanoate | 5.000 |
| Glyceryl stearate | 3.000 |
| PEG-40 stearate | 2.000 |
| Tapioca starch + Aqua | 4.000 |
| Parfum | 0.250 |
| Glycerol + Aqua | 5.000 |
| Aqua + sodium hydroxide | 5.100 |
| Gluconolactone | 2.000 |
| Glycolic acid + Aqua | 10.000 |
| Salicylic acid | 1.000 |
| Decylene glycol | 0.300 |
| Caprylyl glycol | 0.250 |
| Phenoxyethanol | 0.400 |
| Cetearyl alcohol | 3.000 |
| Ammonium Acryloyldimethyltaurate/ | 0.200 |
| VP Copolymer + Aqua | |
| Xanthan gum | 0.500 |
| PEG-150 distearate | 1.000 |
| Aqua + trisodium EDTA | 2.000 |
| Pigments/colorants | As required |
| Water | To 100.000 |

| Example 5 | % by weight |
|---|---|
| Panthenol + Aqua | 0.800 |
| Glycine Soja Germ Extract | 1.000 |
| Hyaluronic acid | 0.250 |
| Glycyrrhiza inflata root extract | 0.050 |
| Dicaprylyl ether | 3.000 |
| Cyclopentasiloxane | 2.000 |
| PEG-100 stearate | 3.000 |
| Sorbitan stearate | 2.000 |
| Distarch phosphate | 4.000 |
| Glycerol + Aqua | 4.000 |
| Lactic acid + Aqua | 2.220 |
| Aqua + sodium hydroxide | 0.240 |
| Glycolic acid + Aqua | 11.450 |
| Caprylyl glycol | 0.250 |
| Phenoxyethanol | 0.400 |
| Xanthan gum | 0.600 |
| PEG-150 distearate | 0.500 |
| Aqua + trisodium EDTA | 1.000 |
| Pigments/colorants | As required |
| Butyl Methoxybenzoylmethane | 1.000 |
| Homosalate | 4.000 |
| Ethylhexyl salicylate | 1.500 |
| Octocrylene | 5.000 |
| Ethylhexyltriazone | 3.000 |
| Bis-Ethylhexyloxyphenol | 2.000 |
| Methoxyphenyl Triazine | |
| Water | To 100.000 |

| Example 6 | % by weight |
|---|---|
| Panthenol + Aqua | 0.800 |
| Ubiquinone (Q10) | 0.030 |
| Tocopheryl acetate | 1.000 |
| Glycyrrhiza inflata root extract | 0.050 |
| Cyclomethicone | 5.000 |
| Glyceryl stearate | 2.000 |
| PEG-40 stearate | 3.000 |
| Wheat starch | 4.000 |
| Glycerol + Aqua | 5.000 |
| Aqua + sodium hydroxide | 0.240 |
| Gluconolactone | 2.000 |
| Glycolic acid + Aqua | 10.000 |
| Salicylic acid | 0.500 |
| Pentylene glycol | 0.900 |
| Phenoxyethanol | 0.500 |
| Behenyl alcohol | 3.000 |
| Hydroxymethylcellulose | 0.100 |
| Xanthan gum | 0.500 |
| PEG-150 distearate | 1.000 |
| Aqua + trisodium EDTA | 2.000 |
| Pigments/colorants | As required |
| Water | To 100.000 |

| Example 7 | % by weight |
|---|---|
| Panthenol + Aqua | 0.800 |
| Isobutylamido Thiazolyl Resorcinol | 0.200 |
| Carnitine | 0.500 |
| Tocopheryl acetate | 1.000 |
| Glycyrrhiza inflata root extract | 0.050 |
| Dimethicone | 1.000 |
| Isodecyl neopentanoate | 2.000 |
| Sodium stearoyl glutamate | 2.000 |
| PEG-100 stearate | 2.000 |
| Sorbitan stearate | 2.000 |
| PEG-40 stearate | 2.000 |
| Rice starch | 4.000 |
| Parfum | 0.250 |
| Glycerol + Aqua | 8.000 |
| Lactic acid + Aqua | 3.330 |
| Aqua + sodium hydroxide | 5.100 |
| Glycolic acid + Aqua | 5.000 |
| Salicylic acid | 0.500 |
| Decylene glycol | 0.300 |
| Caprylyl glycol | 0.250 |
| Alcohol denat. | 5.000 |
| Phenoxyethanol | 0.300 |
| Xanthan gum | 0.400 |
| PEG-150 distearate | 1.000 |
| Aqua + trisodium EDTA | 1.000 |
| Pigments/colorants | As required |
| Water | To 100.000 |

| Example 8 | % by weight |
|---|---|
| Panthenol + Aqua | 0.800 |
| Ubiquinone (Q10) | 0.030 |
| Tocopheryl acetate | 1.000 |
| Glycyrrhetinic acid | 0.050 |
| Hyaluronic acid | 0.250 |
| Glycyrrhiza inflata root extract | 0.050 |
| Dimethicone | 1.000 |
| Dicaprylyl ether | 2.000 |
| Cyclomethicone | 2.000 |
| PEG-100 stearate | 2.000 |
| Glyceryl stearate | 2.000 |
| Silica/talc/kaolin | 2.000 |
| Tapioca starch + Aqua | 2.000 |
| Glycerol + Aqua | 3.000 |
| Aqua + sodium hydroxide | 5.400 |
| Gluconolactone | 2.000 |
| Pentanediol | 0.500 |
| Ethylhexylglycerol | 0.300 |
| Pentylene glycol | 0.700 |
| Phenoxyethanol | 0.600 |
| Stearyl alcohol | 3.000 |
| Xanthan gum | 0.150 |
| PEG-150 distearate | 0.750 |
| Aqua + trisodium EDTA | 2.000 |
| Pigments/colorants | As required |
| Butyl Methoxybenzoylmethane | 1.000 |
| Homosalate | 6.000 |
| Ethylhexyl salicylate | 0.500 |
| Octocrylene | 7.000 |
| Ethylhexyltriazone | 1.500 |
| Bis-Ethylhexyloxyphenol | 3.000 |
| Methoxyphenyl Triazine | |
| Water | To 100.000 |

| Example 9 | % by weight |
|---|---|
| Panthenol + Aqua | 0.800 |
| Tocopheryl acetate | 2.000 |
| Glycyrrhetinic acid | 0.010 |
| Hyaluronic acid | 0.300 |
| Glycyrrhiza inflata root extract | 0.050 |
| Dimethicone | 3.000 |
| Octyl palmitate | 3.000 |
| PEG-100 stearate | 1.000 |
| Glyceryl stearate | 1.000 |
| PEG-40 stearate | 4.000 |
| Rice starch | 2.000 |
| Wheat starch | 2.000 |
| Parfum | 0.250 |
| Glycerol + Aqua | 6.000 |
| Lactic acid + Aqua | 2.220 |
| Aqua + sodium hydroxide | 0.240 |
| Gluconolactone | 2.000 |
| Salicylic acid | 1.800 |
| Ethylhexylglycerol | 0.300 |
| Pentylene glycol | 0.700 |
| Hexanediol | 0.500 |
| Alcohol denat. | 3.000 |
| Phenoxyethanol | 0.400 |
| Cetearyl alcohol | 2.000 |
| Hydroxymethylcellulose | 0.150 |
| Ammonium Acryloyldimethyltaurate/ | 0.150 |
| VP Copolymer + Aqua | |
| Xanthan gum | 0.150 |
| PEG-150 distearate | 1.500 |
| Aqua + trisodium EDTA | 1.000 |
| Water | To 100.000 |

| Example 10 | % by weight |
|---|---|
| Panthenol + Aqua | 0.800 |
| Carnitine | 0.500 |
| Hyaluronic acid | 0.150 |
| Glycyrrhiza inflata root extract | 0.050 |
| Dicaprylyl ether | 1.000 |
| Cyclopentasiloxane | 1.000 |
| Sodium stearoyl glutamate | 2.000 |
| PEG-100 stearate | 2.000 |
| Sorbitan stearate | 1.000 |
| PEG-40 stearate | 1.000 |
| Silica/talc/kaolin | 2.000 |
| Distarch phosphate | 2.000 |
| Glycerol + Aqua | 7.000 |
| Lactic acid + Aqua | 2.220 |
| Aqua + sodium hydroxide | 5.600 |
| Gluconolactone | 2.000 |
| Salicylic acid | 1.800 |
| Caprylyl glycol | 0.500 |
| Pentylene glycol | 0.900 |
| Hexanediol | 0.500 |
| Alcohol denat. | 2.000 |
| Phenoxyethanol | 0.600 |
| Cetyl palmitate | 3.000 |
| Hydroxymethylcellulose | 0.150 |
| Ammonium Acryloyldimethyltaurate/ | 0.150 |
| VP Copolymer + Aqua | |
| Xanthan gum | 0.200 |
| PEG-150 distearate | 0.750 |
| Aqua + trisodium EDTA | 2.000 |
| Pigments/colorants | As required |
| Butyl Methoxybenzoylmethane | 1.000 |
| Ethylhexyl salicylate | 1.500 |
| Octocrylene | 3.000 |
| Bis-Ethylhexyloxyphenol | 2.000 |
| Methoxyphenyl Triazine | |
| Water | 100.000 |

Instructions for the production of all formulations:

Melt fatty phase at 80° C. Disperse starch in the cold water phase and then heat to 90° C. with stirring. Stir the thickener phase into the melted fat phase just before combining the fatty phase and water phase. Combine the phases with stirring and homogenize at 65° C. Add the active substance phase, preservative phase, and perfume phase and homogenize again at 30° C.

1.-10. (canceled)

11. A cosmetic or dermatological O/W emulsion, wherein the emulsion comprises, based on a total weight of the emulsion,
(a) from 3% to 10% by weight of one or more o-hydroxycarboxylic acids and/or one or more β-hydroxycarboxylic acids and/or one or more polyhydroxy acids,
(b) one or more distearates having a melting range in a temperature range of 53-64° C.,
(c) one or more monostearates having a melting range in a temperature range of 40-57° C.,
(d) a water phase
(e) an oil phase
(f) from 0.1% to 2% by weight of one or more starches preswollen at 85-95° C. in an aqueous medium.

12. The emulsion of claim 11, wherein the α-hydroxycarboxylic acid or acids comprise at least one compound of the general formula 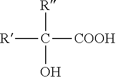
and/or the β-hydroxycarboxylic acid or acids comprise at least one compound of the general formula 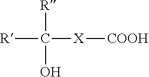
wherein:
X represents an aliphatic CH₂ group, a cycloaliphatic CH group, an aromatic CH group or a CH(OH) group,
R′ and R″ are each independently selected from
(a1) H,
(a2) branched or unbranched C₁₋₂₅ alkyl,
(a3) branched or unbranched C₁₋₂₅ alkyl substituted with one or more carboxyl groups and/or hydroxyl groups and/or aldehyde groups and/or oxo groups (keto groups)
(a4) phenyl,
(a5) phenyl substituted with one or more carboxyl groups and/or hydroxyl groups and/or branched and/or unbranched C₁₋₂₅ alkyl groups,
or wherein
an α-carbon atom of the α-hydroxycarboxylic acid or a β-carbon atom of the β-hydroxycarboxylic acid form together with R′ and X
(a6) an unsubstituted cycloalkyl group having from 3 to 7 ring atoms or
(a7) a cycloalkyl group having from 3 to 7 ring atoms and substituted with one or more carboxyl groups and/or hydroxyl groups and/or oxo groups (keto groups) and/or branched and/or unbranched C₁₋₂₅ alkyl groups,
and wherein the α-hydroxycarboxylic acid or acids and/or the β-hydroxycarboxylic acid or acids may optionally be present as their physiologically tolerated salts and/or ethyl esters and/or methyl esters.

13. The emulsion of claim 11, wherein the α-hydroxycarboxylic acid or acids comprise at least one of citric acid and lactic acid.

14. The emulsion of claim 11, wherein the β-hydroxycarboxylic acid or acids comprise salicylic acid.

15. The emulsion of claim 11, wherein the polyhydroxycarboxylic acid or acids comprise gluconolactone.

16. The emulsion of claim 11, wherein (b) comprises PEG-150 distearate.

17. The emulsion of claim 11, wherein (c) comprises glyceryl stearate.

18. The emulsion of claim 11, wherein (c) comprises PEG-40 stearate.

19. The emulsion of claim 11, wherein the emulsion comprises from 5% to 10% by weight of one or more α-hydroxycarboxylic acids and/or one or more β-hydroxycarboxylic acids and/or one or more polyhydroxy acids.

20. The emulsion of claim 11, wherein the emulsion comprises from 0.001% to 10% by weight of (b).

21. The emulsion of claim 11, wherein the emulsion comprises from 0.05% to 5% by weight of (b).

22. The emulsion of claim 11, wherein the emulsion comprises from 0.001% to 10% by weight of (c).

23. The emulsion of claim 11, wherein the emulsion comprises from 0.05% to 5% by weight of (c).

24. The emulsion of claim 21, wherein the emulsion comprises from 0.1% by weight to 2% by weight of (b) and/or from 0.1% to 2% by weight of (c).

25. A cosmetic or dermatological O/W emulsion, wherein the emulsion comprises, based on a total weight of the emulsion,
(a) from 5% to 10% by weight of one or more α-hydroxycarboxylic acids comprising at least one of citric acid and lactic acid and/or one or more β-hydroxycarboxylic acids comprising salicylic acid and/or one or more polyhydroxy acids comprising gluconolactone,
(b) one or more distearates having a melting range in a temperature range of 53-64° C. and comprising PEG-150 distearate,
(c) one or more monostearates having a melting range in a temperature range of 40-57° C. and comprising glyceryl stearate and/or PEG-40 stearate,
(d) a water phase
(e) an oil phase
(f) from 0.1% to 2% by weight of one or more starches preswollen at 85-95° C. in an aqueous medium.

26. The emulsion of claim 25, wherein one or more α-hydroxycarboxylic acids are present.

27. The emulsion of claim 25, wherein one or more β-hydroxycarboxylic acids are present.

28. The emulsion of claim 25, wherein one or more polyhydroxycarboxylic acids are present.

29. The emulsion of claim 25, wherein the emulsion comprises from 0.1% to 2% by weight of (b) and from 0.1% to 2% by weight of (c).

30. A process for producing a cosmetic or dermatological O/W emulsion, wherein the process comprises combining
(a) from 3% to 10% by weight of one or more o-hydroxycarboxylic acids and/or one or more β-hydroxycarboxylic acids and/or one or more polyhydroxy acids,
(b) one or more distearates having a melting range in a temperature range of 53-64° C.,
(c) one or more monostearates having a melting range in a temperature range of 40-57° C.,
(f) from 0.1% to 2% by weight of one or more starches preswollen at 85-95° C. in an aqueous medium,
water-soluble or hydrophilic constituents being dissolved or dispersed in
(d) a water phase
and oil-soluble or lipophilic constituents being dissolved or dispersed in
(e) an oil phase,
and emulsifying the oil phase and the water phase together, optionally with heating
and with stirring.
